(19) United States
(12) Patent Application Publication
(10) Pub. No.: US 2004/0249188 A1
(43) Pub. Date: Dec. 9, 2004

(54) Process for the preparation of 3-ethoxy-4-(alkoxy carbonyl)-phenyl acetic acid. (an intermediate of repaglinide)

(75) Inventors: Manne Satyanarayana Reddy, Hyderabad (IN); Sajja Eswaraiah, Hyderabad (IN); Anumula Raghupathi Reddy, Hyderabad (IN); Gilla Goverdhan, Hyderabad (IN); Emani Srinivasa Reddy, Hyderabad (IN); Surasani Rajendra, Hyderabad (IN)

Correspondence Address: Ladas & Parry, 26 West 61st Street, New York, NY, 10023, US

(73) Assignees: DR. REDDY'S LABORATORIES LIMITED; DR. REDDY'S LABORATORIES, INC

(21) Appl. No.: 10/856,353

(22) Filed: May 28, 2004

(30) Foreign Application Priority Data
May 29, 2003 (IN) 434/MAS/2003

(51) Int. Cl. C07C 69/76

(52) U.S. Cl. 560/64

(57) ABSTRACT

The present invention relates to an improved and convenient process for the preparation of 3-Ethoxy-4-(alkoxy carbonyl)-phenyl acetic acid, which can be represented by formula (Ia) where R¹ represents ethyl or methyl. Specifically the present invention relates to an improved process for the preparation of compound of formula

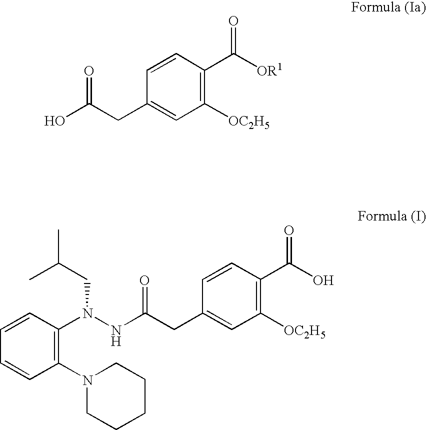

(Ia), which is the key intermediate for Repaglinide of formula (I), by the process, which involves non-hazardous raw materials with an easy handling, and cost effective process

### FIELD OF THE INVENTION

The present invention relates to an improved and convenient process for the preparation of 3-Ethoxy-4-(alkoxy carbonyl)-phenyl acetic acid, which can be represented by formula (Ia) where R¹ represents ethyl or methyl. Specifically the present invention relates to an improved process for the preparation of compound of formula (Ia), which is the key intermediate for Repaglinide of formula (I), by the process, which involves non-hazardous raw materials with an easy handling, and cost effective process

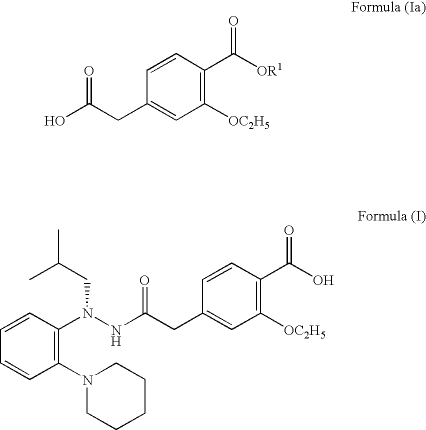

Repaglinide is a known oral anti-diabetic drug used for the treatment of diabetes, called Type-2 diabetes. It may be used alone or with metformin.

### BACKGROUND OF THE INVENTION

In Journal of Medicinal Chemistry 1998 Vol.41, No.26,5219 the process for the preparation of hypoglycemic benzoic acid derivatives was disclosed. Specifically disclosed the process for the preparation of 3-Ethoxy-4-(ethoxy carbonyl)-phenyl acetic acid. The process of the preparation of 3-Ethoxy-4-(ethoxy carbonyl)-phenyl acetic acid comprises of reacting 2-Hydroxy-4-methyl-benzoic acid with ethyl bromide in the presence of K₂CO₃ in acetone at 150° C. for 30 hrs in autoclave to give Ethyl-2-ethoxy-4-methyl-benzoate. The obtained compound was reacted with NBS in the presence of 2,2¹-azo-bis-(isobutyronitril) in CCl₄ to yield Ethyl-4-bromomethyl-benzoate. And this 4-bromomethyl ester is reacted with NaCN in the presence of N-benzyl-tri-n-butylammonium chloride in water and dichloromethane at 20° C. for 43 hrs to give Ethyl-4-cyanomethyl-2-ethoxy-benzoate. The cyano methyl ester was treated with gaseous HCl in ethanol at reflux to yield Ethyl-2-ethoxy-4-ethoxycarbomethyl-benzoate. The obtained diester was hydrolyses with 2N NaOH in ethanol at 23-25° C. for 1.5 hr to yield 3-Ethoxy-4-(ethoxy carbonyl)-phenyl acetic acid.

The above process described for the preparation of 3-Ethoxy-4-(ethoxy carbonyl)-phenyl acetic acid has some disadvantages to perform in large scale as it involves lacrimetic chemicals like ethyl bromide, which is difficult to handle in commercial scale. The usage of carbon tetra chloride as a solvent is yet again set back in the process, since it is class-I solvent, and usage of ethanol as a solvent is also a set back in the process as the recovery and reuse of said solvent is not feasible in scale up. And the process has another disadvantage is all the reactions have longer hour maintenance, higher temperature and HCl gas passing at refluxing temperature. Still the process has another disadvantage is the formation of diacid impurity during the preparation of formula IV and I. Hence, the process renders with high cost and is not suitable for commercial production.

WO 01/35900 A2 describes the process for the preparation of 3-ethoxy-4-(ethoxy carbonyl) phenyl acetic acid. This process comprises of reacting 4-methyl salicylic acid with ethylbromide in dimethyl sulfoxide at 35-40° C. to give ethyl-2-ethoxy-4-methyl benzoate. The obtained compound was reacted with n-butyl lithium in a solution of diisopropyl amine in tetra hydrofuran and hexamethyl phosphoramide, then reacted with carbon dioxide at −75° C. to give 3-ethoxy-4-(ethoxy carbonyl) phenyl acetic acid.

The above said patent process for the preparation of 3-ethoxy-4-(ethoxy carbonyl) phenyl acetic acid has some disadvantages to perform in large scale as it involves lacrimatic chemicals like ethyl bromide, moisture sensitive and fire hazardous chemicals like n-butyl lithium. And the process has another disadvantage is operational bottleneck like −75° C. temperature. Still the process has another disadvantage is the usage of tetrahydrofuran and dimethylsufoxide as the recovery and reuse of said solvents is not feasible in scale up. The said process mentioned in WO 01/35900 A2 is suffering with scale up operations, so there is need to develop to overcome the above said disadvantages.

These forgoing problems, directed us towards the present invention, which is the convenient and economic process for the preparation of compound of the formula (Ia), which is the key intermediate for the preparation of Repaglinide of the formula (I), is a known oral anti-diabetic drug used for the treatment of diabetes. The present invention involves a cheaper and easy handling chemicals like diethyl sulfate instead of ethyl bromide, cyclohexane (class-II) solvent instead of CCl₄ (class-I), cheaper solvent methanol instead of ethanol, cheaper phase transfer catalyst tetra butyl ammonium bromide instead of N-benzyl-tri-n-butylammonium chloride and dry HCl gas passing also avoided as mentioned in the prior art. The present process an advantageous over prior art references that all stages are obtained with good yield and purity.

The product obtained in the present process is having high yield than prior art and the process is cost effective, Eco-friendly and easily scalable.

### SUMMARY OF THE INVENTION

The present invention relates to an improved and convenient process for the preparation of 3-Ethoxy-4-(alkoxy carbonyl)-phenyl acetic acid of formula (Ia), which is the key intermediate for preparation of Repaglinide of formula (I) is used for the treatment of anti diabetes.

The process of the present invention comprises the esterification and etherficaton of (2-Hydroxy-4-methyl) benzoic acid of formula (II) with diethyl sulfate using potassium carbonate in toluene as a solvent to give Ethyl-2-ethoxy-4-methyl-benzoate of formula (III). Which on allylic bromination with NBS in the presence of AIBN as a catalyst in cyclohexane resulted Ethyl-4-bromo methyl-2-ethoxy-benzoate of formula (IV). The bromo methyl compound on cyanation with sodium cyanide in the presence of tetra butyl ammonium bromide as a phase transfer catalyst in dichloro-methane and water as a solvent afforded Ethyl-4-cyanomethyl-2-ethoxy-benzoate of formula (V). Which on hydrolysis in the presence of sodium hydroxide in water resulted (4-carboxy-3-ethoxy-phenyl) acetic acid of formula (IV). The obtained diacid compound on esterfication in the presence of trimethyl amine in toluene afforded the Alkyl-2-ethoxy-4-alkoxy carbonyl methyl-benzoate of formula (III), the di ester compound selectively hydrolyses with sodium hydroxide in methanol to give 3-Ethoxy-4-(alkoxy carbonyl)-phenyl acetic acid of formula (Ia).

The process of the present invention is cost effective and eco-friendly over prior art procedures.

### DETAILED DESCRIPTION OF THE INVENTION

Accordingly, the present invention provides an improved and convenient process for the preparation of 3-Ethoxy-4-(alkoxy carbonyl)-phenyl acetic acid of formula (Ia).

The process of the present invention is schematically represented as follows.

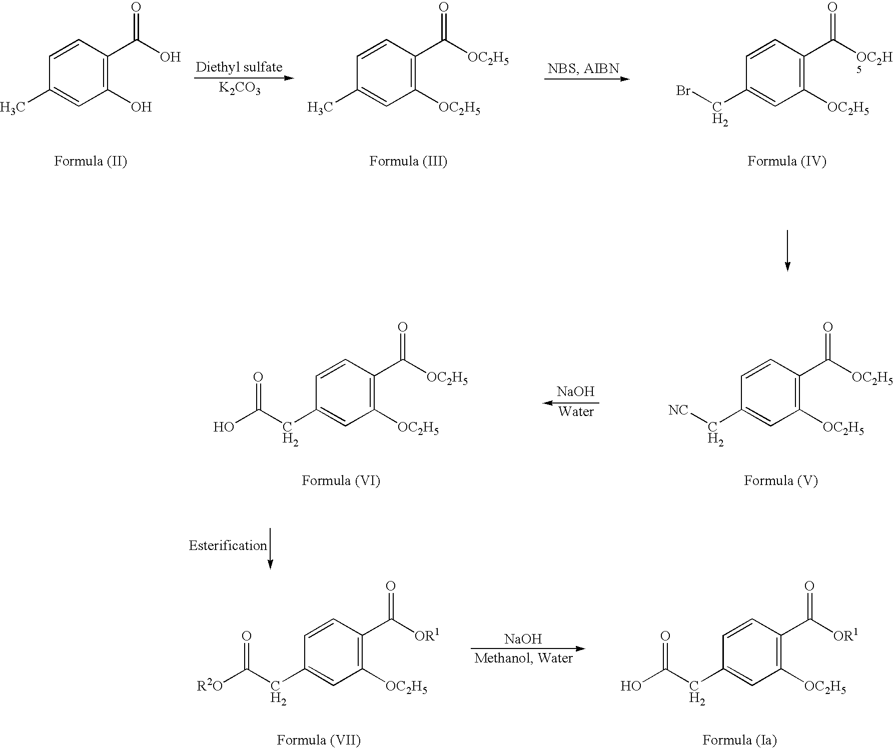

The present invention provides a process for the preparation of compound of formula (Ia) as shown in the above scheme, which comprises.

Preparation of compound of formula (III) from compound of formula (II) by esterification and etherification with diethyl sulfate.

Transforming the compound of formula (III) into compound of formula (IV) involves allylic bromination with NBS in the presence of AIBN.

Transforming the compound of formula (IV) into compound of formula (V) involves cyanation with NaCN.

Preparation of compound of formula (VI) from compound of formula (V) by hydrolysis.

Preparation of compound of formula (VII) from compound of formula (VI) by esterification.

Conversion of compound of formula (VII) into compound of formula (Ia) by selective hydrolysis of ethoxy carbonyl methyl group.

Another aspect of the process of present invention includes preparation of intermediate described by general formula (VI)

The process to prepare compound of formula (III) includes, reacting the carboxy and hydroxy groups of compound of formula (II) with diethylsulfate in the presence of suitable base such as potassium carbonate, sodium carbonate, triethyl amine, potassium-t-butoxide and like in suitable solvent such as C₁-C₄ ketone, toluene, benzene, cyclohexane and like. The temperature ranges from 80-110° C., preferably 100-110° C.

The reagents used in the above process may range from equimolar to 5 mole ratio. The duration of reaction may range from 15-35 hr, preferably from 20-30 hrs.

Preparation of compound of formula (IV) involves allylic bromination of compound of formula (III) with suitable allylic brominating reagents like N-bromo succinimide, dibromo dimethyl hydentoin in the presence suitable free radical initiators such as 2,2¹-azo-bis-(isobutyronitrile), di benzoyl peroxide and like in suitable solvents. Such as chloroform, CCl₄, cyclohexane and dichloromethane, preferably in cyclohexane. The temperature ranges from 25° C. to reflux temperature of the solvent used preferably 75-85° C. The duration of the reaction may range from 3 hr to 10 hr, preferably 3 hr to 5 hr. The reagents used in the process may range from 1 mole to 2 mole ratio.

Transformation of compound of formula (IV) into compound of formula (V) involves cyanation with sodium cyanide in suitable solvent. Such as mixture of water and organic solvents like dichloromethane, toluene, benzene, chloroform ethyl acetate, in the presence of suitable phase transfer catalyst such as tetra butyl ammonium halide, benzyl trimethyl ammonium halide, N-benzyl-tri-n-butyl ammonium halide and the like, where halide represents chlorine, bromine or iodine.

The quantity of water may range from 0.5 times to 4 times and quantity of solvent may range from 3 times to 10 times.

In preparation of compound of formula (VI) includes, hydrolysis of cyano ester compound of formula (V) with suitable acidic reagents such as hydrochloric acid, hydrobromic acid, sulfuric acid and the like, suitable basic reagents such as sodium hydroxide, potassium hydroxide, sodium hydride, potassium hydride, preferably in sodium hydroxide and the like, in suitable solvent. Such as water, C₁-C₄ alcohol, dichloromethane, chloroform, acetonitrile and the like at suitable temperature may range from 25° C. to reflux temperature of the solvent used, preferably 80° C. to 100° C.

The reagents used in the above process may range from 2 moles to 4 mole ratio, preferably 3 mole ratio.

Preparation of compound of formula (VII), involves esterification with suitable reagents such as ethyl bromide, ethyl chloride, diethyl sulfate, methyl bromide, methyl chloride, dimethyl sulfate and the like. In the presence of suitable base such as potassium carbonate, sodium carbonate, sodium bicarbonate, potassium bicarbonate, triethyl amine and the like in suitable solvent such as toluene, C₁-C₄ ketone, cyclohexane, benzene and the like.

The reagents used in the above process may range from 1 mole to 4 mole ratio, preferably 3 mole ratio. The temperature may ranges from 25° C. to reflux temperature of the solvent used, preferably 90-110° C.

The process for the preparation of compound of formula (Ia) includes, selective hydrolysis of compound of formula (VII) with suitable reagents, may be acidic or basic. Acidic reagents such as hydrochloric acid, hydrobromic acid, sulfuric acid, acetic acid and the like, basic reagents such as sodium hydroxide, potassium hydroxide, sodium hydride, potassium hydride, and the like, in suitable solvent. Such as, C₁-C₄ alcohol, acetonitrile, C₁-C₄ ketone and the like or mixture of water and organic solvents like C₁-C₄ alcohol, acetonitrile, C₁-C₄ ketone, preferably mixture of water and methanol, at suitable temperature may range from 0° C. to reflux temperature of solvent used, preferably 10° C. to 15° C.

The reagents used in the above process may range from 0.8 moles to 1.2 mole ratio, preferably equimolar ratio, the duration of the reaction may range from 1 hr to 20 hr preferably 1 hr to 2 hr.

The above process, includes isolation of compound by pH adjustment with suitable acidic reagents such as hydrochloric acid, hydrobromic acid, sulfuric acid and the like, preferably hydrochloric acid. The pH may range from 1 to 6, preferably 1 to 3.

The process described in the present invention is demonstrated in examples illustrated below. These examples are provided an illustration only and therefore should not be construed as limitation to the scope of invention.

### EXAMPLE 1

### Preparation of Ethyl-2-ethoxy-4-methyl benzoate (III)

Added 260 ml (3.0 mol) of diethyl sulfate to the mixture of 1000 ml of toluene, 272.5 g (3.0 mol) of potassium carbonate and 100 g (1.0 mol) of 2-Hydroxy-4-methyl-benzoic acid at 25-45° C. for 1 hr to 1 hr 30 min. Heated the reaction mass to azeotropic reflux for 20-30 hr. Cooled the reaction mixture to 25-35° C. and collected the unwanted material by filtration, washed the unwanted material with 500 ml of toluene. Combined filtrate was washed with water (1×1000 ml and 1×500 ml) at 80-85° C. Total organic layer was concentrated to residue under vacuum at below 70° C. The yield of the title compound is 128 g (94.11%).

The obtained product was characterised by analytical techniques like IR, Mass and ¹H-NMR

### EXAMPLE 2

### Preparation of Ethyl-4-bromo methyl-2-ethoxy benzoate (IV)

To the suspension of 700 ml of cyclo hexane, 86 g (1.0 mol) of N-bromo succinimide and 3 g (0.03 mol) of AIBN was added 100 g (1.0 ml) of Ethyl-2-ethoxy-4-methyl benzoate. Heated the resulted mass to reflux and stirred for 3-5 hr. Cooled to 50-60° C. and charged 200 ml of water, then stirred for 10-15 min. Separated the aqueous phase and organic phase. The organic phase was washed with 200 ml of 5% hydrose followed by water (200 ml) at 50-60° C. Total organic phase was distilled completely under vacuum at below 60° C. Cooled to 25-35° C. and the title compound was isolated from residue with 100 ml n-Heptane at 0-5° C. The obtained compound was recrystallised from 400 ml of n-Heptane. Dried the compound under reduced pressure at 45-50° C. The yield of the recrystallised compound is 59.2 g (43%).

The obtained product was characterised by analytical techniques like IR, Mass and ¹H-NMR

### EXAMPLE 3

### Preparation of Ethyl-4-cyanomethyl-2-ethoxy benzoate (V)

A solution of 350 ml of dichlro methane and 50 g (1.0 mol) of Ethyl-4-bromo methyl-2-ethoxy benzoate was added slowly to a suspension of 30 ml of water, 13 g (1.5 mol) of sodium cyanide and 2.5 g of tetra butyl ammonium bromide at temperature of 10-15° C. Then raised the reaction mass temperature to 25-35° C. and stirred for 25-35 hr. After completion of reaction separated the aqueous phase and organic phase. Washed the organic phase with water (1×100 ml and 1×50 ml). Distilled off the organic phase under reduced pressure at below 45° C. The titled compound was isolated from residue with 100 ml of isopropyl alcohol at a temperature of 0-5° C. Dried the compound under reduced pressure at 35-40° C. The yield of the obtained product is 32.7 g (80.3%).

The obtained product was characterised by analytical techniques like IR, Mass and ¹H-NMR

### EXAMPLE 4

### Preparation of (4-Carboxy-3-ethoxy-phenyl) acetic acid (VI)

Charged 50 g (1.0 mol) of Ethyl-4-cyanomethyl-2-ethoxy benzoate to a solution of 250 ml of water and 26 g (3.0 mol) of sodium hydroxide. The resulted reaction mixture was heated to reflux for 1½ hr to 2 hr. Cooled the reaction mass to 25-35° C. and adjust the pH to around 2 with hydrochloric acid at 25-35° C., stirred for 45-60 minutes. Filtered the compound and washed with 100 ml of water. Dried the product at 70-80° C. The yield of the title compound is 46.1 g (96%).

The obtained product was characterised by analytical techniques like IR, Mass and ¹H-NMR

### EXAMPLE 5

### Preparation of Ethyl-2-ethoxy-4-ethoxy carbonyl methyl benzoate (VII)

Added 94 ml (3.0 ml) of triethylamine and 88 ml (3.0 ml) of diethyl sulfate to a suspension of 250 ml of toluene and 50 g (1.0 mol) of (4-carboxy-3-ethoxy-phenyl) acetic acid at temperature of 25-35° C. The resulted reaction mass was heated to reflux for 1-2 hr. Charged 500 ml of water into reaction mass at 80-85° C., and stirred for 15-30 minutes. Separated the aqueous phase and organic phase. Washed the organic phase with 250 ml of water at 80-85° C. and distilled under reduced pressure at below 70° C. Cooled to 25-35° C. and unloaded the residue. The yield of the obtained product is 58 g (92.8%).

The obtained product was characterised by analytical techniques like IR, Mass and ¹H-NMR

### EXAMPLE 6

### Preparation of 3-Ethoxy-4-(ethoxy carbonyl)-phenyl acetic acid (VI)

A solution of 15 ml of water and 1.4 g (0.98 mol.) of sodium hydroxide was added slowly to a slowly to a solution of 25 ml of methanol and 10 g (1.0 mol) of Ethyl-2-ethoxy-4-ethoxy carbonyl methyl benzoate at a temperature of 10-15° C. Stirred for 1-2 hrs at 10-15° C., then the solvent was distilled off from reaction solution under reduced pressure at below 60° C. Cooled to 25-35° C. and charged 20 ml of water and 20 ml of toluene. Then stirred for 15-30 min, separated the aqueous phase and organic phase. Washed the aqueous phase with 20 ml of toluene. Aqueous layer pH was adjusted to 34 with hydrochloric acid at temperature of 0-5° C. Product was extracted with toluene (2×30 ml) from acidified aqueous layer at temperature of 50-60° C. Washed the total organic phase with water (3×30 ml), and concentrated under reduced pressure at below 70° C. The title compound was isolated from residue with 20 ml of cyclohexane at a temperature of 10-15° C. Dried the product at 45-50° C. The yield is 7 g (77.8%).

The obtained product was characterised by analytical techniques like IR, Mass and ¹H-NMR

### EXAMPLE 7

### Preparation of Methyl-2-ethoxy-4-methoxy carbonyl methyl benzoate (VII)

Charged 30 g (1.0 mol) of (4-Carboxy-3-ethoxy-phenyl) acetic acid to a suspension of 300 ml of toluene, 39 ml (3.0 mol) of dimethyl sulfate and 55.8 g (3.0 mol) of potassium carbonate. Resulted reaction mass was heated to reflux for 1 hr to 2 hr and charged 300 ml of water into reaction mass at 80-85° C., stirred for 15-30 min. Separated the aqueous phase and organic phase. Washed the organic phase with 150 ml of water at 80-85° C. Organic phase was distilled under reduced pressure at below 70° C. The yield of the title product is 31.8 g (94.2%).

The obtained product was characterised by analytical techniques like IR, Mass and ¹H-NMR

### EXAMPLE 8

### Preparation of 3-Ethoxy-4-(methoxy carbonyl)-phenyl acetic acid (Ia)

Charged 20 g (1.0 mol.) of methyl-2-ethoxy-4-methoxy carbonyl methyl benzoate and 50 ml of methanol to a solution of 30 ml of water and 3 g (0.95 mol) of sodium hydroxide at temperature of 25-35° C. Stirred the reaction mass for 1 hr to 2 hr at 25-35° C. Distilled off the solvent from reaction solution under reduced pressure. Cooled to 25-35° C. and charged 40 ml of water and 40 ml of toluene then stirred for 15-30 minutes. Separated the aqueous phase and organic phase. Washed the aqueous phase with 40 ml of toluene. Aqueous layer pH was adjusted to 3-4 with hydrochloric acid at a temperature of 25-35° C. Product was extracted with toluene (2×40 ml) from acidified aqueous layer at a temperature of 30° C. Washed the total organic phase with water (2×40 ml), and concentrated under reduced pressure. The title compound was isolated from residue with 40 ml of cyclohexane at a temperature of 25-35° C. Dried the product at 45-50° C. The yield is 10 g (53%).

The obtained product was characterised by analytical techniques like IR, Mass and ¹H-NMR

1. A compound of formula (Ia) 3-Ethoxy-4-(alkoxy carbonyl)-phenyl acetic acid and its improved process 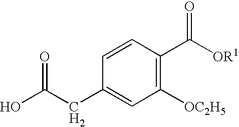
where R¹ represents either ethyl or methyl group which comprises:
(a) reacting a compound of formula (II) with suitable ethylating reagent diethyl sulfate, ethyl chloride preferably diethyl sulfate in the presence of suitable base in a suitable solvent; 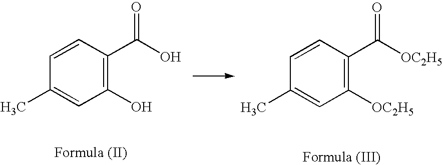
(b) reacting a compound of formula (III) with suitable allylic brominating reagents in presence of suitable free radical initiator in suitable solvent such as cyclohexane; 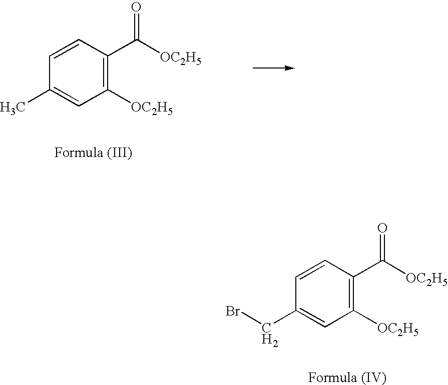
(c) reacting a compound of formula (V) with suitable hydrolysing reagent in suitable solvent medium; 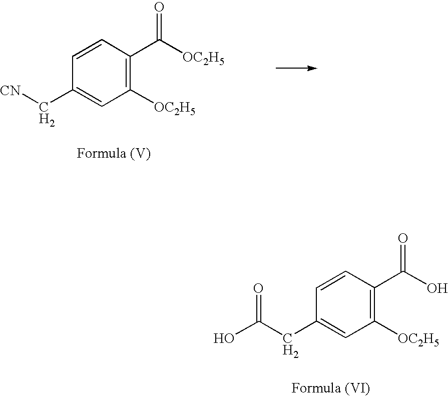
(d) reacting a compound of formula (VI) with suitable esterfication reagent such as diethyl sulfate, dimethyl sulfate, ethyl bromide, ethyl chloride in presence of suitable base in a suitable solvent. 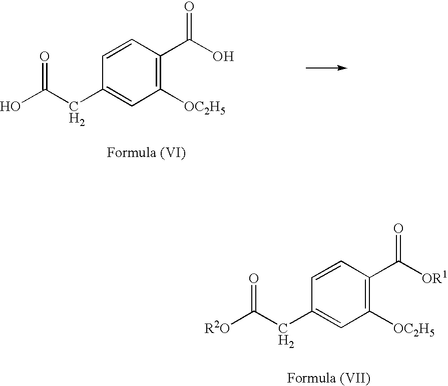
where R¹ and R² represents either ethyl or methyl groups,
(e) reacting a dimethyl ester compound of formula (VII) with suitable hydrolysing reagent in suitable solvent medium.

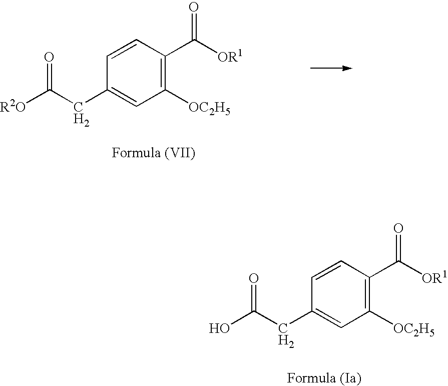

2. The process according to claim 1 of step (a), wherein the suitable ethylating reagent is diethyl sulfate.

3. The process according to claim 1 of step (a), where in the suitable base is potassium carbonate, sodium carbonate, potassium-t-butoxide or triethylamine, preferably potassium carbonate in suitable solvent.

4. The process according to claim 3, where in the suitable solvent is C₁-C₄ ketone, toluene, benzene, cyclohexane, preferably toluene.

5. The process according to claim 3, wherein the mole ratio of base may range from 1 mole to 5 mole, preferably 3.0 moles.

6. The process according to claim 2, wherein the mole ratio of ethylating reagent is 1 mole to 5 mole, preferably 3.0 moles.

7. The process for the preparation of compound of formula (IV) in the presence of a solvent cyclohexane at suitable temperature.

8. The process according to claim 7, where in the suitable temperature may range from 25° C. to reflux temperature of the used solvent, preferably 75-85° C.

9. The process for the preparation of compound of formula (VI) by hydrolysis of compound of formula (V) in the presence of hydrochloric acid or hydrobromic acid or sulfuric acid or aqueous sodium hydroxide, preferably aqueous sodium hydroxide at a suitable temperature.

10. The process according to claim 9, the temperature may range from 25° C. to 100° C. preferably 80-100° C.

11. The process for the preparation of diester compound of formula (VII) by esterification of compound of formula (VI) by using esterifying agent such as esterification reagent is ethyl bromide, ethyl chloride, diethyl sulfate, methyl bromide, methyl chloride or dimethyl sulfate, preferably diethyl sulfate or dimethyl sulfate under suitable base in the presence of a suitable solvent.

12. The process according to claim 11, where in the suitable base is potassium carbonate, sodium carbonate, sodium bicarbonate, potassium bicarbonate or triethyl amine, and preferably potassium carbonate.

13. The process according to claim 11, the suitable solvent is toluene, benzene, C₁-C₄ ketone or cyclohexane, preferably toluene.

14. The process according to claim 11, where in the mole ratio of esterification reagent and base may range from 1 mole to 4 mole ratio, preferably 3 mole ratio.

15. The process for the preparation of compound of formula (VII) according to claim 11 is, 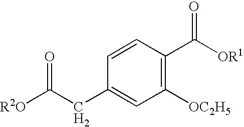
where R¹ and R² represents ethyl or methyl group.

16. The process according to claim 1 of step (e), conversion of dimethyl ester compound of formula (VII) by hydrolysis in a suitable hydrolysing reagent such as acidic or basic either hydrochloric acid, hydrobromic acid, sulfuric acid, acetic acid, sodium hydroxide potassium hydroxide, sodium hydride or potassium hydride, preferably sodium hydroxide in suitable solvent.

17. The process according to claim 16, where in the suitable solvent in C₁-C₄ alcohol, C₁-C₄ketone, acetonitrile, or mixture of water and above solvent, preferably mixture of water and methanol.

18. The process according to claim 16, the mole ratio of base is may range from 0.8 moles to 1.2 ratios, preferably equi molar ratio.

19. The process for the preparation of 3-Ethoxy-4-(methoxy carbonyl)-Phenyl acetic acid (an intermediate of Repaglinide) is substantially as here in described and exemplified.
